SEQUENCE LISTING (20 sequences)

SEQ ID NO: 1 - DNA, 588 bp, Artemisia annua L.
atggacaattttgtttctgtttttccatacaataattcctcatcttcaacttcaccacatctatcacttaacttgatgaacgattactcgtatagtagtgatcaaaacaatacgtacgatcacctagaggacgatcatgagcatggtttagttgagaaaaaccctatttctagctcagaagaagttgtgctagatcatgtttctcccacgagtagcggtttaggtaatagtgatggaaatgatatgagttcggcttccgggtctcggaagattagtattaagaaaggcgaaaagaagattagaaaacctaagtgtgcgtttcaaacgagaagccaagttgatatacttgatgatggttatagatggaggaagtatggccaaaaggctgttaagaataataagttcccaaggagctattaccgctgtacgtatcaaggatgtaacgtgaagaaacaagtccaaaggctatcaaaagacgagggagtcgttgtgacaacttacgaaggaatgcattcacatccaatcgagaaatctaccgataactttgagcatattttgactcaaatgcaaatctattcttcatgctag

SEQ ID NO: 2 - protein, 195 aa, Artemisia annua L.
MDNFVSVFPYNNSSSSTSPHLSLNLMNDYSYSSDQNNTYDHLEDDHEHGLVEKNPISSSEEVVLDHVSPTSSGLGNSDGNDMSSASGSRKISIKKGEKKIRKPKCAFQTRSQVDILDDGYRWRKYGQKAVKNNKFPRSYYRCTYQGCNVKKQVQRLSKDEGVVVTTYEGMHSHPIEKSTDNFEHILTQMQIYSSC

SEQ ID NO: 3 - DNA, 549 bp, Mentha haplocalyx Briq
atggataactattcccaaccttcttcgtcttcatcaactcttgcacaaagctctcatatatccatgctcaacatgatgatgaactctcaaccacacgatcatcaactattccagcatcttgatcagaataatggacacgtgggcttcatcccgtccgttgaaaataatgatcataagcctagctccgccgtcgagggtggtggtggtccggagccggaaaacgaggcggaaggcggcaagagaaagggggagaagaagtctaagaaacctaggtttgccttccaaacaagaagccaagttgatatacttgatgatggatataggtggaggaaatatggtcaaaaggcagtcaagaacaatagatttcccaggagctactacagatgcacacaacaaggttgcaatgtaaagaaacaagtgcaaaggctatcaaaagatgaagggatagtggtgactacttatgaaggagtccattctcatccaatccaaaaatctacagacaattttgaccacattcttagtcaaatgcagatttatactgccttctaa

SEQ ID NO: 4 - protein, 182 aa, Mentha haplocalyx Briq
MDNYSQPSSSSSTLAQSSHISMLNMMMNSQPHDHQLFQHLDQNNGHVGFIPSVENNDHKPSSAVEGGGGPEPENEAEGGKRKGEKKSKKPRFAFQTRSQVDILDDGYRWRKYGQKAVKNNRFPRSYYRCTQQGCNVKKQVQRLSKDEGIVVTTYEGVHSHPIQKSTDNFDHILSQMQIYTAF

SEQ ID NO: 5 - DNA, 558 bp, Mentha spicata Linn
atggataactattcccaaccttcttcttcatcaagtctcgcacaaagctctcatctatccatgctcaacatgatgatgaactcccaaccacacgatcatcaactattccaacatcttgatcagaataatggacacgtgggcttcatcccatccgttgaaaataatgatgatcataagtctagttccgccgccgttgaggttgagggtggtggtggtgcggaggcggaaaacgaggcggaaggcggcaagagaaagggggagaagaagtccaagaaacctaggtttgccttccaaacaagaagccaagttgatatacttgatgatggttataggtggaggaaatatggtcaaaaggctgtcaagaacaatagatttcccaggagctactacagatgcacacatcaaggctgcaatgtaaagaaacaagtgcagaggctatcaaaagacgaaggaatagtggtgactacttatgaaggcgtccattctcatcctatccaaaaatctaccgacaattttgaccacatccttagtcaaatgcagatttatactgccttctaa

SEQ ID NO: 6 - protein, 185 aa, Mentha spicata Linn
MDNYSQPSSSSSLAQSSHLSMLNMMMNSQPHDHQLFQHLDQNNGHVGFIPSVENNDDHKSSSAAVEVEGGGGAEAENEAEGGKRKGEKKSKKPRFAFQTRSQVDILDDGYRWRKYGQKAVKNNRFPRSYYRCTHQGCNVKKQVQRLSKDEGIVVTTYEGVHSHPIQKSTDNFDHILSQMQIYTAF

SEQ ID NO: 7 - DNA, 24 bp, Artificial Sequence
  AaWRKY75b-FP Primer
atggacaattttgtttctgttttt

SEQ ID NO: 8 - DNA, 23 bp, Artificial Sequence
  AaWRKY75b-RP Primer
ctaaaacaaaggtggatcttgta

SEQ ID NO: 9 - DNA, 32 bp, Artificial Sequence
  BamHI-AaWRKY75b-FP Primer
cgggatccatggacaattttgtttctgttttt

SEQ ID NO: 10 - DNA, 31 bp, Artificial Sequence
  AaWRKY75b-XbaI-RP Primer
gctctagactaaaacaaaggtggatcttgta

SEQ ID NO: 11 - DNA, 22 bp, Artificial Sequence
  35SF Primer
gaagatgcctctgccgacagtg

SEQ ID NO: 12 - DNA, 31 bp, Artificial Sequence; identical to SEQ ID NO: 10
  AaWRKY75b-RP Primer for PCR detection of transgenic Artemisia annua plants

SEQ ID NO: 13 - DNA, 25 bp, Artificial Sequence
  MsWRKY75b-FP Primer
atggataactattcccaaccttctt

SEQ ID NO: 14 - DNA, 25 bp, Artificial Sequence
  MsWRKY75b-RP
ttagaaggcagtataaatctgcatt

SEQ ID NO: 15 - DNA, 25 bp, Artificial Sequence
  MhWRKY75b-FP Primer
atggataactattcccaaccttctt

SEQ ID NO: 16 - DNA, 25 bp, Artificial Sequence
  MhWRKY75b-RP Primer
ttagaaggcagtataaatctgcatt

SEQ ID NO: 17 - DNA, 33 bp, Artificial Sequence
  BamHI-MsWRKY75b-FP Primer
cgggatccatggataactattcccaaccttctt

SEQ ID NO: 18 - DNA, 33 bp, Artificial Sequence
  MsWRKY75b-XbaI-RP Primer
gctctagattagaaggcagtataaatctgcatt

SEQ ID NO: 19 - DNA, 33 bp, Artificial Sequence; identical to SEQ ID NO: 17
  BamHI-MhWRKY75b-FP Primer

SEQ ID NO: 20 - DNA, 33 bp, Artificial Sequence; identical to SEQ ID NO: 18
  MhWRKY75b-XbaI-RP Primer

(19) United States
(12) Patent Application Publication
(10) Pub. No.: US 2021/0010018 A1
(43) Pub. Date: Jan. 14, 2021

(54) NUCLEOTIDE SEQUENCE AND USE THEREOF IN INCREASING THE DENSITY OF SECRETORY GLANDULAR TRICHOMES IN PLANTS

(71) Applicants: SHANGHAI JIAO TONG UNIVERSITY, Shanghai (CN); Suzhou Tangen Biotechnology Co., Ltd, Suzhou (CN)

(72) Inventors: Kexuan TANG, Shanghai (CN); Lihui XIE, Shanghai (CN); Xueqing FU, Shanghai (CN); Wei QIN, Shanghai (CN); Ling LI, Shanghai (CN); Hang LIU, Shanghai (CN); Tiantian CHEN, Shanghai (CN); Hongmei QIAN, Shanghai (CN); Xiaofen SUN, Shanghai (CN)

(73) Assignees: SHANGHAI JIAO TONG UNIVERSITY, Shanghai (CN); Suzhou Tangen Biotechnology Co., Ltd, Suzhou (CN)

(21) Appl. No.: 17/030,385

(22) Filed: Sep. 24, 2020

Related U.S. Application Data
(63) Continuation-in-part of application No. PCT/CN2019/072349, filed on Jan. 18, 2019.

(51) Int. Cl. C12N 15/82; C07K 14/415

(52) U.S. Cl. CPC C12N 15/8262; C12N 15/8205; C07K 14/415

(57) ABSTRACT

The present invention discloses a nucleotide sequence and use thereof in increasing the density of secretory glandular trichomes in plants. The nucleotide sequence is selected from the group consisting of: 1) a nucleotide sequence according to any one of SEQ ID NOs: 1, 3 and 5; 2) a nucleotide sequence derived from the nucleotide sequence according to any one of SEQ ID NOs: 1, 3 and 5 through substitution, deletion or addition of one or more nucleotides; 3) a nucleotide sequence having at least 80% homology with any one of SEQ ID NOs: 1, 3 and 5. The present invention significantly increases the density of secretory glandular trichomes by transferring any one of the above nucleotide sequences into the plants by means of genetic engineering. Therefore, the invention shows great potential in insect resistance and production of specific metabolites, and has extremely high practical application value.

### CROSS REFERENCE TO THE RELATED APPLICATIONS

This application is continuation-in-part application of the International Application No. PCT/CN2019/072349, filed on Jan. 18, 2019, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named “GBSHJL003-Sequence Listing-20200922.txt”, dated Sep. 22, 2020 and is 10,753 bytes in size.

### TECHNICAL FIELD

The invention relates to the technical field of plant genetic engineering, in particularly relates to a nucleotide sequence and use thereof in increasing the density of secretory glandular trichomes in plants.

### BACKGROUND

Glandular trichomes are protruding structures derived from plant epidermal cells. According to whether they can secrete and store secondary metabolites, glandular trichomes are divided into non-secretory and secretory glandular trichomes. Secretory glandular trichomes are distributed on the epidermis of aerial organs in about 30% of vascular plants in nature, for example, Lamiaceae plants such as Mentha, Lavandula angustifolia, Rosmarinus officinalis and Ocimum basilicum; Asteraceae plants such as Artemisia annua and Helianthus annuus; Solanaceous plants such as Nicotiana tabacum and Solanum lycopersicum; Leguminosae plants such as Medicago truncatula and Medicago saliva; and Moraceae plants such as Humulus lupulus and Cannabis saliva, etc.; all of these have secretory glandular trichomes. Secretory glandular trichomes not only help plants resist diseases and insect pests as well as ultraviolet rays, but also secrete a lot of chemicals. These chemicals mainly include terpenes, methylbenzenes, flavonoids, methyl ketones, acyl carbohydrates, etc. These chemicals can be used in pharmaceuticals, production of pesticides and essential oils, etc., thus have high commercial value. Therefore, secretory glandular trichomes are also called plant factories. Among them, Artemisia annua, also known as Artemisia apiacea, is an annual traditional Chinese herbal plant in the genus Artemisia, family Asteraceae. Artemisia annua has secretory glandular trichomes and T-shape non-secretory glandular trichomes. Artemisinin is a sesquiterpene lactone compound containing a peroxy bridge structure. Artemisinin and derivatives thereof are mainly used to treat malaria. Artemisinin-based combination therapy is the most effective method recommended by the World Health Organization to treat falciparum malaria. However, artemisinin is only synthesized and stored in the secretory glandular trichomes of Artemisia annua.

The number of secretory glandular trichomes varies in plants. In some crops with important economic value, insufficient quantity and density of secretory glandular trichomes restricts the production of secondary metabolites.

Therefore, those skilled in the art are committed to developing a method for increasing the density of secretory glandular trichomes in plants by means of transgenic technology.

### SUMMARY

In view of the above defects of prior art, the technical problem to be solved by the present invention is how to increase the density of secretory glandular trichomes in plants.

In order to achieve the above objective, the present invention provides a nucleotide sequence, wherein the nucleotide sequence is selected from the group consisting of:

1) a nucleotide sequence according to any one of SEQ TD NOs: 1, 3 and 5;

2) a nucleotide sequence derived from the nucleotide sequence according to any one of SEQ ID NOs: 1, 3 and 5 through substitution, deletion or addition of one or more nucleotides;

3) a nucleotide sequence having at least 80% homology with any one of SEQ Ill NOs: 1, 3 and 5.

In another aspect, the present invention provides an amino acid sequence, wherein the amino acid sequence is selected from the group consisting of:

1) an amino acid sequence according to any one of SEQ ID NOs: 2, 4 and 6;

2) an amino acid sequence derived from the amino acid sequence according to any one of SEQ ID NOs: 2, 4 and 6 through substitution, deletion or addition of one or more amino acids;

3) an amino acid sequence having at least 80% homology with any one of SEQ ID NOs: 2, 4 and 6.

In another aspect, the present invention also provides a use of the nucleotide sequence or the amino acid sequence as described above in increasing the density of secretory glandular trichomes in plants.

Further, the plants described above are one or more of Lamiaceae plants, Leguminosae plants, Asteraceae plants and Moraceae plants.

Specifically, the Lamiaceae plants include Mentha, Lavandula angustifolia and Rosmarinus officinalis, the Asteraceae plants include Artemisia annua, and the Moraceae plants include Cannabis saliva.

Further, the plants described above are one or more of: Mentha haplocalyx Briq, Mentha spicata Linn, Cynara cardunculus var. scolymus, Helianthus annuus, Solanum lycopersicum, Solarium pennellii, Solarium tuberosum, Cannabis saliva, Lavandula angustifolia, Rosmarinus officinalis, Ocimum basilicum, Pelargonium hortorum, Chrysanthemum cinerariaefolium, Humulus lupulus and Medicago sativa.

In another aspect, an embodiment of the present invention provides a transgenic method for increasing the density of secretory glandular trichomes in plants, including the following steps:
(1) obtaining target gene through gene cloning, the target gene being any one of the nucleotide sequences as mentioned above;
(2) constructing a plant expression vector containing the target gene;
(3) transforming Agrobacterium tumefaciens with the plant expression vector containing the target gene, to obtain an Agrobacterium tumefaciens strain which carries the plant expression vector containing the target gene;
(4) transforming plants with the Agrobacterium tumefaciens strain which carries the plant expression vector containing the target gene, and obtaining transgenic plants integrated with the target gene through PCR detection;
(5) calculating the glandular trichomes density on the leaves of the transgenic plants integrated with the target gene to obtain plants with increased glandular trichomes density.

Further, in step (1), gene cloning comprises the steps of: extracting total RNA of the plant genome, synthesizing cDNA by reverse transcription, performing PCR amplification with the primers as shown in SEQ ID NOs: 7 and 8 and performing sequence analysis to obtain the target gene.

Further, in step (2), constructing a plant expression vector containing the target gene comprises the steps of: amplifying the sequence of the target gene with high-fidelity enzyme, introducing BamHI and XbaI restriction sites before and after the target gene respectively, connecting the target gene to the vector with ligase, transforming host cell, picking monoclonal colony, and extracting plasmids for PCR detection and restriction enzyme digestion verification.

Further, in step (3), transforming Agrobacterium tumefaciens comprises the steps of: transferring the plant expression vector containing the target gene into Agrobacterium tumefaciens through freeze-thaw method, and performing PCR verification.

Further, in step (4), transforming comprises the steps of: pre-culturing explants; co-culturing Agrobacterium tumefaciens and the explants; and screening for the regenerated plants containing the target gene in antibiotic-containing culture medium.

Further, in an embodiment, the plants described above are Artemisia annua.

According to the present invention, the plants used in the transgenic method for increasing the density of secretory glandular trichomes in plants include but are not limited to Artemisia annua, also include Cannabis sativa, Mentha, Lavandula angustifolia and Rosmarinus officinalis.

Further, the target gene described above is any one of: AaWRKY75b gene of Artemisia annua, MhWRKY75b) gene of Mentha haplocalyx Briq and MsWRKY75b gene of Mentha spicata Linn.

Further, the gene sequence of AaWRKY75b of Artemisia annua is shown in SEQ ID NO: 1; the gene sequence of MhWRKY75b of Mentha haplocalyx Briq is shown in SEQ ID NO: 3; and the gene sequence of MsWRKY75b of Mentha spicata Linn is shown in SEQ ID NO: 5.

Further, the amino acid sequence encoded by AaWRKY75b gene of Artemisia annua is shown in SEQ IS NO: 2; the amino acid sequence encoded by MhWRKY75b gene of Mentha haplocalyx Briq is shown in SEQ ID NO: 4; the amino acid sequence encoded by MsWRKY75b gene of Mentha spicata Linn is shown in SEQ ID NO: 6;

Further, in step (1), the primer sequences are shown in SEQ ID NOs: 7 and 8.

Further, cDNA is synthesized under the action of reverse transcriptase PowerScript.

Further, pre-culturing explants comprises the steps of: soaking the seeds of Artemisia annua in 75% ethanol for 1 min and then in 20% NaClO for 20 min; rinsing the seeds with sterile water for 3-4 times; drying the moisture on the surface of the seeds with sterile absorbent paper; inoculating the seeds in hormone-free MS solid medium; culturing the seeds at 25° C. for 16 hours light/8 hours dark to obtain sterile seedlings of Artemisia annua; after the seedlings growing to about 5 cm, cutting the sterile seedlings to get leaf explants for transformation.

Further, co-culturing Agrobacterium tumefaciens and the explants comprises the steps of: transferring the leaf explants to the co-culture medium supplemented with acetosyringone (AS); adding the activated ½ MS bacterial solution of Agrobacterium tumefaciens containing the plant expression vector of the target gene; fully contacting the leaf explants with the bacterial solution; and culturing in the dark at 28° C. for 3 days.

Further, screening for antibiotic-resistant regenerated plants comprises the steps of: transferring the leaf explants that had been co-cultured for 3 days into the germination screening medium which contains 6-benzylaminopurine (6-BA), naphthaleneacetic acid (NAA), hygromycin (Hyg) and carbenicillin (Cb); culturing at 25° C. for 16 hours light/8 hours dark; performing subculture ever two weeks; obtaining Hyg-resistant cluster buds after 2-3 subcultures; cutting and transferring the well-grown resistant cluster buds to rooting medium; culturing to rooting; and obtaining Hyg-resistant regenerated Artemisia annua plants.

Further, the method further comprises the step of:

(6) determining the artemisinin content in transgenic Artemisia annua plants by HPLC-ELSD, and screening for transgenic Artemisia annua plants with increased artemisinin content.

In embodiments of the present invention, overexpression of Artemisia annua AaWRKY75b gene or genes having 80% or more homology therewith (such as Mentha haplocalyx Briq MhWRKY75b gene or Mentha spicata Linn MsWRKY75b gene) in plants with secretory glandular trichomes by transgenic means significantly increased the secretory glandular trichomes density in transgenic plants, thereby increasing the content of secondary metabolites such as artemisinin, which has extremely high practical application value. For the plant Artemisia annua, transgenic plants with significantly increased density of secretory glandular trichomes on leaf surface were obtained, thereby enabling to transform the secretory glandular trichomes of Artemisia annua into high-yield biochemical factory. Moreover, it also showed by HPLC-ELSD that the artemisinin content of the plants with increased density of secretory glandular trichomes was also significantly increased, showing great potential in insect resistance and production of specific metabolites, which has extremely high practical application value.

The concept, embodiments and technical effects of the present invention will be further described below in conjunction with the accompanying drawings to fully understand the purpose, features and effects of the present invention.

### BRIEF DESCRIPTION OF THE DRAWINGS

FIG. 1 is a graph comparing glandular trichomes density on leaves of AaWRKY75b-transgenic Artemisia annua plants and wild-type Artemisia annua plants according to an embodiment of the present invention;

FIG. 2 is a graph showing the statistical results of the glandular trichomes density on leaves of AaWRKY75b-transgenic Artemisia annua plants and wild-type Artemisia annua plants according to an embodiment of the present invention;

FIG. 3 is a graph showing the artemisinin content in AaWRKY75b-transgenic Artemisia annua plants and wild-type Artemisia annua plants according to an embodiment of the present invention;

FIG. 4 is a graph comparing the glandular trichomes density on leaves of MhWRKY75b-transgenic or MsWRKY75b-transgenic Artemisia annua plants and wild-type Artemisia annua plants according to an embodiment of the present invention;

FIG. 5 is a graph showing the statistical results of the glandular trichomes density in MhWRKY75b-transgenic Artemisia annua plants and wild-type Artemisia annua plants according to an embodiment of the present invention;

FIG. 6 is a graph showing the statistical results of the glandular trichomes density on leaves of MsWRKY75b-transgenic Artemisia annua plants and wild-type Artemisia annua plants according to an embodiment of the present invention.

### DETAILED DESCRIPTION OF THE EMBODIMENTS

The technical content of the present invention will be further described below with reference to the drawings and examples. The following examples will help those skilled in the art to further understand the present invention, but do not limit the present invention in any form. In the following examples, the experimental procedures without specific conditions generally follow the conventional conditions, such as those described in J. Sambrook, et al, Molecular cloning: A laboratory manual, Cold Spring Harbor Laboratory Press, 1989, or the conditions recommended by manufacturers.

The Agrobacterium tumefaciens EHA105 involved in the present invention has been published in “Huang YAK Jiang Xiliang, Tian Yunlong, Guo Ping, Zhu Changxiong, The Study of Agrobacterium tumefaciens-mediated genetic transformation of Trichoderma harzianum, Chinese Journal of Bioengineering, 2008, 28(3): 38-43”. The Agrobacterium tumefaciens EHA105 is commercially available, for example, it can be purchased from CAMBIA, Australia under Catalog No. Gambar1.

### EXAMPLE 1

Extraction of Total RNA from Artemisia annua

The Artemisia annua leaves were quickly ground into powder in liquid nitrogen. Total RNA of Artemisia annua was extracted following the instructions of the RNA Kit of TIANGEN. Part of the RNA solution obtained was taken to determine the total RNA quality by agarose gel electrophoresis and to determine the RNA concentration by spectrophotometer, and the remaining part was stored at −80° C.

### EXAMPLE 2

Cloning of AaWRKY75b) Gene in Artemisia annua

Using the total RNA extracted in Example 1 as template, cDNA was synthesized with PowerScript reverse transcriptase. Gene-specific primers were designed based on the sequence of Artemisia annua AaWRKY75b gene (GenBank accession number: KX465129.1). The primer sequences were shown in Table 1. The AaWRKY75b gene ofArtemisia annua was amplified from the total cDNA by PCR. The PCR reaction system was shown in Table 2. The amplified products were recovered by gel electrophoresis, then DNA sequencing was performed. After sequencing, the full-length coding sequence of the gene was obtained, as shown in SEQ ID NO: 1, in which the start codon was ATG and the stop codon was TAG. The protein sequence was thus deduced, as shown in SEQ ID NO: 2.

TABLE 1
PCR primers for AaWRKY75b gene cloning

| Name | Sequence (5′→3′) | ID Number |
|---|---|---|
| AaWRKY75b- | ATGGACAATTTTGTTTCTGTTTTT | SEQ ID |
| FP | | NO: 7 |
| AaWRKY75b- | CTAAAACAAAGGTGGATCTTGTA | SEQ ID |
| RP | | NO: 8 |

TABLE 2
PCR reaction system

| | |
|---|---|
| cDNA of Artemisia annua | 1 μL |
| 10 × KOD Plus Buffer | 5 μL |
| dNTP | 5 μL |
| MgSO4 | 2 μL |
| AaWRKY75b-FP | 1 μL |
| AaWRKY75b-RP | 1 μL |
| KOD Plus | 1 μL |
| ddH2O | 34 μL |
| Total volume | 50 μL |

### EXAMPLE 3

Construction of Plant Overexpression Vector Containing AaWRKY75b Gene

AaWRKY75b gene sequence was amplified with high-fidelity enzyme. The sequences of the amplification primer were shown in Table 3. BamHI restriction site was introduced into the forward primer, and XbaI restriction site was introduced into the reverse primer. The amplified sequence was ligated to pCAMbia 1305.1 vector (purchased from Youbio) with ligase. Sequencing was performed to confirm the correctness of the gene.

TABLE 3
PCR primers in the construction of AaWRKY75b-pCAMbia
1305.1 vector

| Name | Sequence (5′→3′) | ID Number |
|---|---|---|
| BamHI- | CGGGATCCATGGACAATTTTGTTTCTGTTTTT | SEQ ID NO: 9 |
| AaWRKY75b-FP | | |
| AaWRKY75b- | GCTCTAGACTAAAACAAAGGTGGATCTTGTA | SEQ ID NO: 10 |
| XbaI-RP | | |

### EXAMPLE 4

Obtaining Agrobacterium tumefaciens Engineering Bacteria Containing the AaWRKY75b Overexpression Vector

The plant overexpression vector containing the AaWRKY75b gene obtained in Example 3 was transferred into Agrobacterium tumefaciens by freeze-thaw method, and PCR verification was performed. The verification primers were shown in SEQ ID NOs: 11 and 12. Agrobacterium tumefaciens strains containing the plant overexpression vector of AaWRKY75b gene were obtained.

### EXAMPLE 5

### Agrobacterium tumefaciens-mediated Transformation of AaWRKY75b Gene Into Artemisia annua

(1) Pre-Culture of Explants

The Artemisia annua seeds were soaked in 75% ethanol for 1 min and then in 20% NaClO for 20 min. Then the seeds were rinsed with sterile water for 3-4 times. The moisture on the surface of the seeds was dried with sterile absorbent paper. The seeds were inoculated in hormone-free MS solid medium, and cultured at 25° C. for 16 hours light/8 hours dark to obtain sterile seedlings of Artemisia annua. After the seedlings growing to about 5 cm, the sterile seedlings were cut to get leaf explants for transformation.

(2) Co-Culture of Agrobacterium tumefaciens and the Explants

The leaf explants were transferred to co-culture medium supplemented with acetosyringone (AS) (½ MS+AS 100 μmol/L). The activated ½MS bacterial solution of Agrobacterium tumefaciens containing the plant overexpression vector of AaWRKY75b gene was added. The leaf explants were fully contacted with the bacterial solution and then cultured in the dark at 28° C. for 3 days. The leaf explants added with ½ MS liquid medium suspension of Agrobacterium tumefaciens without the target gene were used as controls.

(3) Screening for Antibiotic-Resistant Regenerated Plants

The leaf explants co-cultured for 3 days were transferred into the germination screening medium supplemented with 6-benzylaminopurine (6-BA), naphthaleneacetic acid (NAA). hygromycin (Hyg) and carbenicillin (Cb) (MS+6-BA 0.5 mg/L NAA+0.05 mg/L+Hyg (overexpression) 50 mg/L+Cb 500 mg/L), and cultured at 25° C. for 16 hours light/8 hours dark. Subculture was performed every two weeks. Hyg-resistant cluster buds were obtained after 2-3 subcultures. The well-grown resistant cluster buds were cut and transferred to rooting medium, and cultured to rooting. Then Hyg-resistant regenerated Artemisia annua plants were obtained.

### EXAMPLE 6

PCR Detection of Transgenic Artemisia annua Plants

According to the 35S promoter region at the upstream of the target gene expression cassette and the AaWRKY75b gene, the forward primer (35SF: GAAGATGCCTCTGCCGACAGTG; SEQ ID NO: 11) and the reverse primer (AaWRKY75b -RP: GCTCTAGACTAAAACAAAGGTGGATCTTGTA; SEQ ID NO: 12) were designed to detect the target gene. The results showed that specific DNA fragments can be amplified using the designed PCR specific primers. While using non-transformed Artemisia anima genomic DNA as a template, no fragment was amplified.

In this example, the plant expression vector was transformed into Agrobacterium tumefaciens to obtain an Agrobacterium tumefaciens strain containing plant overexpression vector of AaWRKY75b for transforming Artemisia annua. The Agrobacterium tumefaciens strain constructed was used to transform Artemisia annua. Transgenic Artemisia annua plants were obtained by PCR detection. Transgenic Artemisia anima plants can be directly used for screening Artemisia annua plants with increased secretory glandular trichomes density and increased artemisinin content.

### EXAMPLE 7

Calculating the Epidermal Glandular Trichomes Density and Total Number of Glandular Trichomes in Transgenic Artemisia annua Plants

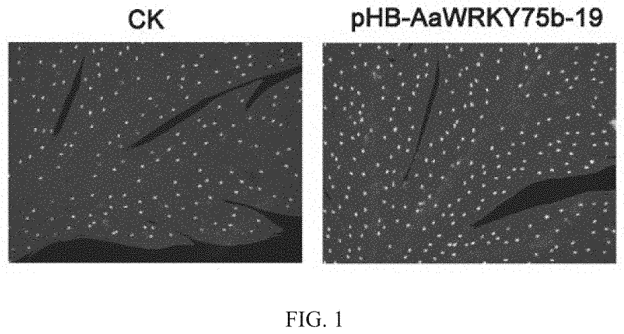

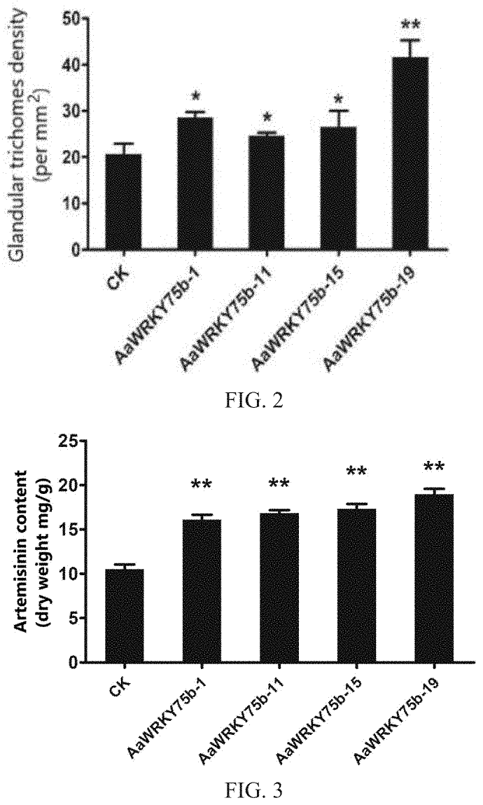

The leaves of non-transgenic Artemisia annua plants and AaWRKY75b overexpressing transgenic Artemisia annua plants were observed with Olympus BX51 microscope under the excitation light of 450 nm-480 nm. Random samples were taken at 5 different sites on two kinds of leaves with same size respectively. ImageJ software is used to measure the total leaf area of Artemisia annua plants and to calculate the density of glandular trichomes. The results were shown in FIGS. 1 and 2, wherein CK represents the wild-type control. The glandular trichomes density of the leaves of AaWRKY75b transgenic Artemisia annua plants was significantly higher than that of wild-type Artemisia annua plants, with a highest density of 41.59 per mm², which was about twice that of wild-type Artemisia annua plants (20.65 per mm² for wild-type Artemisia annua plants)

### EXAMPLE 8

Determination of Artemisinin Content in Transgenic Artemisia annua by HPLC-ELSD

(1) Conditions and System Applicability of HPLC-ELSD and Preparation of Standard Solutions

HPLC: Water alliance 2695 system was chosen, and C-18 reverse-phase silica gel column was chosen as chromatographic column (SymmetryShield TM C18, 5 μm, 250×4.6 mm, Waters). The mobile phase was methanol: water, and the volume ration of methanol : water was 70:30. The column temperature was 30° C. The flow rate was 1.0 mL/min. Sample loading volume was 10 μL. Sensitivity (AUFS)=1.0, and number of theoretical plates calculated according to artemisinin peak was not less than 2000.

ELSD: Water alliance 2420 system was chosen. The evaporative light-scattering detector drift tube temperature was 40° C. The amplification factor (gain) was 7, and the carrier gas pressure was 5 bar.

2.0 mg of artemisinin standard substance (Sigma) vas precisely weighed and completely dissolved in 1 mL of methanol, and a 2 mg/mL artemisinin standard solution was prepared, which was stored at −20° C. until use.

In the present invention, when the mobile phase was methanol: water (70%: 30%), the retention time of artemisinin was 5.1 min, and the peak was in good shape. The number of theoretical plates calculated according to artemisinin peak was not less than 2000.

(2) Standard Curve

2 μL, 4 μL, 6 μL, 8 μL, and 10 μL of control solutions were loaded under corresponding chromatogaphic conditions. The spectrum and chromatographic parameters were recorded. Regression analysis of peak area (Y) versus standard substance content (X, μg) was conducted. In this example, artemisinin exhibits a good log-log linear relationship in the range of 4-20 g. The log-log linear regression equation of the artemisinin standard substance is: Y=1.28e+000X+4.71e+000, R²=0.979546.

(3) Sample Preparation and Artemisinin Content Determination

A total of 2 g fresh Artemisia annua leaves were taken from the upper, middle and lower parts of the Artemisia annua plants and dried in a 45° C. oven to constant weight. The dried leaves were taken from the dried branches and then ground into powder. About 0.1 g of dry powder was weighed into a 2 mL Eppendorf tube. 2 mL of ethanol was added. The solution was treated with 40 W ultrasonic wave for 30 min, and centrifuged at 5000 rpm for 10 min. The supernatant was filtered with a 0.22 μm filter. The filtrate obtained can be used in HPLC-ELSD to determine the content of artemisinin.

The artemisinin content was determined by HPLC-ELSD. The volume of the sample loaded was 20 μL. According to the linear regression equation, the artemisinin content (mg) corresponding to the peak area was obtained. The artemisinin content (mg) was divided by the dry weight of Artemisia annua leaf sample (g) to calculate the artemisinin content in the Artemisia annua plants. The results were shown in FIG. 2. The artemisinin content of the AaWRKY75b-transgenic Artemisia annua plants was significantly correlated with the density of glandular trichomes. With the increase of secretory glandular trichomes density, the content of artemisinin increased to 18.8 mg/g, which was about 1.8 times that of wild type (10.5 mg/g).

### EXAMPLE 9

Sequence Homology Alignment of Artemisia annua AaWRKY75b Gene

Nucleotide sequence homology alignment of AaWRKY75b gene sequence as shown in SEQ ID NO: 1 was performed in NCBI database. The results showed that AaWRKY75b gene sequence was 89% homologous with Probable WRKY transcription factor 75 of Cynara cardunculus var scolymus; 86% homologous with Probable WRKY transcription factor 75 of Helianthus annuus; 86% homologous with WRKY transcription factor 75 of Solanum lycopersicum; 86% homologous with Probable WRKY transcription factor 75 of Solanum pennellii; 85% homologous with WRKY1 gene of Solanum tuberosum; 80% homologous with homologous MsWRKY75b gene of Mentha spicata Linn (GenBank accession number: KT372786.1); and 80% homologous with homologous MhWRKY75b gene of Mentha haplocalyx Briq (as shown in SEQ ID NO: 3).

### EXAMPLE 10

Extraction of Total RNA from Mentha spicata Linn and Mentha haplocalyx Briq Plants

The specific procedures were carried out according to the steps in Example 1.

### EXAMPLE 11

Cloning of MhWRKY75b Gene of Mentha spicata Linn and MsWRKY75b Gene of Mentha haplocalyx Briq

The specific procedures were carried out according to the steps in Example 2, wherein the primers used were shown in Table 4. The full-length coding sequence of Mentha spicata Linn MsWRKY75b gene obtained was shown in SEQ ID NO: 3, and the deduced protein sequence thereof was shown in SEQ ID NO: 4. The full-length coding sequence of Mentha haplocalyx Briq MhWRKY75b) gene obtained was shown in SEQ ID NO: 5, and the deduced protein sequence thereof was shown in SEQ ID NO: 6.

TABLE 4
PCR primers used for MhWRKY75b gene and MsWRKY75b gene
cloning

| Name | Sequence (5′→3′) | ID Number |
|---|---|---|
| MsWRKY75b-FP | ATGGATAACTATTCCCAACCTTCTT | SEQ ID NO: 13 |
| MsWRKY75b-RP | TTAGAAGGCAGTATAAATCTGCATT | SEQ ID NO: 14 |
| MhWRKY75b-FP | ATGGATAACTATTCCCAACCTTCTT | SEQ ID NO: 15 |
| MhWRKY75b-RP | TTAGAAGGCAGTATAAATCTGCATT | SEQ ID NO: 16 |

### EXAMPLE 12

Construction of Plant Overexpression Vectors Containing MsWRKY75b Gene or MhWRKY75b Gene

The specific procedures were carried out according to the steps in Example 3, wherein the primers used were shown in Table 5. The plant overexpression vectors obtained were MhWRKY75b-pCAMbia 1305.1 vector and MsWRKY75b-pCAMbia 1305.1 vector.

TABLE 5
PCR primers in the construction of MhWRKY75b-pCAMbia 1305.1
vector and MsWRKY75b-pCAMbia 1305.1 vector

| Name | Sequence (5′→3′) | ID Number |
|---|---|---|
| BamHI-MsWRKY75b-FP | CGGGATCCATGGATAACT | SEQ ID NO: 17 |
| | ATTCCCAACCTTCTT | |
| MsWRKY75b-XbaI-RP | GCTCTAGATTAGAAGGCA | SEQ ID NO: 18 |
| | GTATAAATTCTGCATT | |
| BamHI-MhWRKY75b-FP | CGGGATCCATGGATAACT | SEQ ID NO: 19 |
| | ATTCCCAACCTTCTT | |
| MhWRKY75b-XbaI-RP | GCTCTAGATTAGAAGGCA | SEQ ID NO: 20 |
| | GTATAAATCTGCATT | |

### EXAMPLE 13

Obtaining Agrobacterium tumefaciens Engineering Bacteria Containing the MhWRKY75b Gene or MsWRKY75b Gene Overexpression Vector

The specific procedures were carried out according to the steps in Example 4. The verification primers were shown in SEQ ID NOs: 11 and 18.

### EXAMPLE 14

Agrobacerium tumefaciens-mediated Transformation of MsWRKY75b Gene or MhWRKY75b Gene into Artemisia annua

The specific procedures were carried out according to the steps in Example 5.

### EXAMPLE 15

PCR Detection of Artemisia annua Plants Transformed with MsWRKY75b Gene or MhWRKY75b Gene

The specific procedures were carried out according to the steps in Example 6. The verification primers were shown in SEQ ID NOs: 11 and 18.

### EXAMPLE 16

Calculating the Epidermal Glandular Trichomes Density and Total Number of Glandular Trichomes in MsWRKY75b-Transgenic or MhWRKY75b-Transgenic Artemisia annua Plants

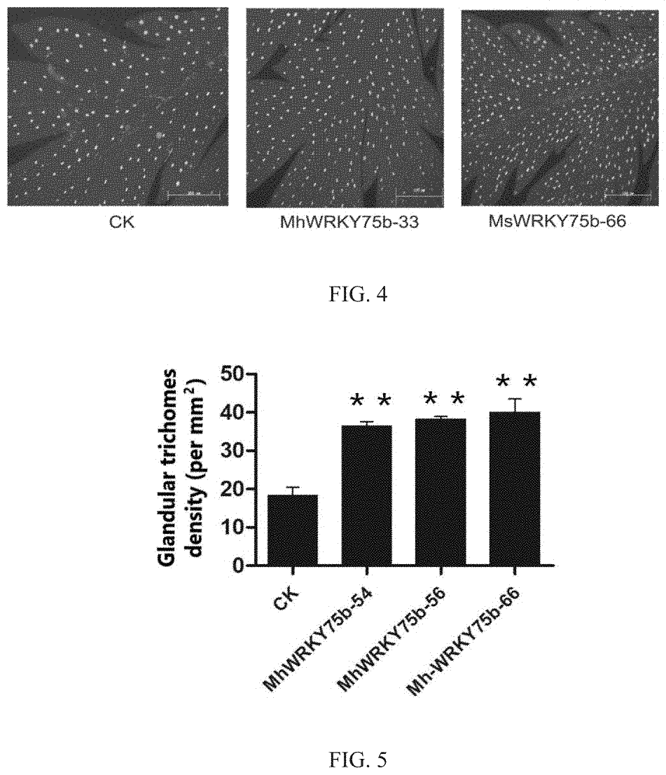

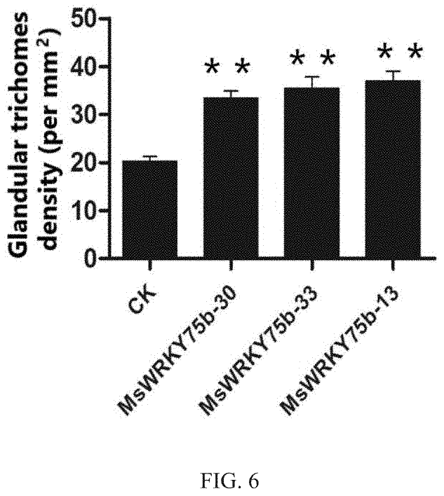

The specific procedures were carried out according to the steps in Example 7. The results were shown in FIGS. 4, 5 and 6, wherein CK represents the wild-type control. The glandular trichomes density of the leaves of MsWRKY75b-transgenic Artemisia annua plants was significantly higher than that of the wild-type Artemisia annua plants, with a highest density of 38.65 per mm², which was about 1.9 times that of wild type (20.34 per mm²). The glandular trichomes density of the leaves of MhWRKY75b-transgenic Artemisia annua plants was significantly higher than that of the wild-type Artemisia annua plants, with the highest density of 36.45 per mm², which was about 1.8 times that of wild type (20.10 per mm²).

The preferred embodiments of the present invention have been described in detail above. It should be understood that those skilled in the art can make various modifications and changes according to the concept of the present invention without creative work. Therefore, any technical solution that can be obtained by those skilled in the art based on the concept of the present invention in combination with prior art through logical analysis, reasoning, or limited experiments should be within the scope of the present claims.

What is claimed is:

1. A nucleotide sequence, wherein the nucleotide sequence is selected from a group consisting of:
1) a nucleotide sequence set forth in any one of SEQ ID NO: 1, SEQ ID NO: 3 and SEQ ID NO: 5;
2) a nucleotide sequence derived from the nucleotide sequence set forth in any one of SEQ ID NO: 1, SEQ ID NO: 3 and SEQ ID NO: 5 through substitution, deletion or addition of one or more nucleotides;
3) a nucleotide sequence having at least 80% homology with any one of SEQ ID NO: 1, SEQ ID NO: 3 and SEQ 1D NO: 5.

2. An amino acid sequence, wherein the amino acid sequence is selected from a group consisting of:
1) an amino acid sequence set forth in any one of SEQ ID NO: 2, SEQ ID NO: 4 and SEQ ID NO: 6;
2) an amino acid sequence derived from the amino acid sequence set forth in any one of SEQ ID NO: 2, SEQ ID NO: 4 and SEQ ID NO: 6 through substitution, deletion or addition of one or more amino acids;
3) an amino acid sequence having at least 80% homology with any one of SEQ ID NO: 2, SEQ ID NO: 4 and SEQ 1D NO: 6.

3. A method of using the nucleotide sequence according to claim 1, wherein the nucleotide sequence is used for increasing a density of secretory glandular trichomes in plants.

4. The method according to claim 3, wherein the plants are one or more of: Mentha haplocalyx Briq, Mentha spicata Linn, Cynara cardunculus vat: scolymus, Helianthus annuus, Solanum lycopersicum, Solanum pennellii, Solanum tuberosum, Cannabis saliva, Lavandula angustifolia, Rosmarinus officinalis, Ocimum basilicum, Pelargonium hortorum, Chrysanthemum cinerariaefolium, Humulus lupulus and Medicago sativa.

5. A transgenic method for increasing a density of secretory glandular trichomes in plants, including the following steps:
(1) obtaining a target gene through gene cloning, wherein the target gene is the nucleotide sequences according to claim 1;
(2) constructing a plant expression vector containing the target gene;
(3) transforming Agrobacterium tumefaciens with the plant expression vector containing the target gene, to obtain an Agrobacterium tumefaciens strain carrying the plant expression vector containing the target gene;
(4) transforming the plants with the Agrobacterium tumefaciens strain carrying the plant expression vector containing the target gene, and obtaining transgenic plants integrated with the target gene through PCR detection;
(5) calculating the density of the secretory glandular trichomes on leaves of the transgenic plants integrated with the target gene to obtain plants with increased glandular trichomes density.

6. The transgenic method according to claim 5, wherein in step (1), the gene cloning comprises the steps of: extracting total RNA of a plant genome, synthesizing cDNA by reverse transcription, performing PCR amplification with primers set forth in SEQ 1D NO: 7 and SEQ ID NO: 8, and performing sequence analysis to obtain the target gene.

7. The transgenic method according to claim 5, wherein in step (2), constructing the plant expression vector containing the target gene comprises the steps of: amplifying a sequence of the target gene with a high-fidelity enzyme, introducing BamHI and XbaI restriction sites before and after the target gene respectively, connecting the target gene to a vector with a ligase, transforming a host cell, picking a monoclonal colony, and extracting plasmids for PCR detection and restriction enzyme digestion verification.

8. The transgenic method according to claim 5, wherein in step (3), transforming the Agrobacterium tumefaciens comprises the steps of: transferring the plant expression vector containing the target gene into the Agrobacterium tumefaciens through freeze-thaw method, and performing PCR verification.

9. The transgenic method according to claim 5, wherein in step (4), transforming comprises the steps of: pre-culturing explants; co-culturing the Agrobacterium tumefaciens strain carrying the plant expression vector containing the target gene and the explants; and screening for regenerated plants containing the target gene in an antibiotic-containing culture medium.

10. The transgenic method according to claim 5, wherein the plants are Artemisia annua, and the transgenic method further comprises the step of:
(6) determining an artemisinin content in transgenic Artemisia annua plants by HPLC-ELSD, and screening for transgenic Artemisia annua plants with an increased artemisinin content.
